(19) United States
(12) Patent Application Publication
(10) Pub. No.: US 2013/0218295 A1
(43) Pub. Date: Aug. 22, 2013

(54) CONTROL SYSTEMS AND METHODS FOR GAIT DEVICES

(71) Applicant: SpringActive, Inc., (US)

(72) Inventor: Matthew Aaron Holgate, Chandler, AZ (US)

(73) Assignee: SpringActive, Inc., Tempe, AZ (US)

(21) Appl. No.: 13/767,945

(22) Filed: Feb. 15, 2013

Related U.S. Application Data
(60) Provisional application No. 61/600,141, filed on Feb. 17, 2012.

(51) Int. Cl. A61F 2/72

(52) U.S. Cl. CPC A61F 2/72
USPC 623/25

(57) ABSTRACT

Methods for controlling gait devices include measuring kinematic and/or loading states of limb or robotic segments; conditioning the resulting state measurement by any combination or order of integration, differentiation, filtering, and amplification; transforming conditioned state measurements by coordinate transformation; optionally conditioning the transformed state measurements a second time in a manner similar to the first conditioning step; and using the transformed or conditioned transformed state measurements as independent variables in a predetermined reference function to calculate a desired reference command for any number of actuators.

### CROSS-REFERENCE TO RELATED APPLICATION

This application claims benefit of priority to U.S. Provisional Application Ser. No. 61/600,141, filed Feb. 17, 2012. The aforementioned priority application is incorporated herein by reference in its entirety.

### BACKGROUND OF THE INVENTION

1. Field of the Invention

The field of the present invention is control systems and methods for gait devices, and particularly control systems and methods for prosthetic, orthotic, and robotic gait devices.

2. Background

Many control systems and methods have been designed for prosthetic, orthotic, and robotic gait devices. Nonetheless, there is still a need for control systems and methods that processes user signals quickly and accurately, while providing smooth and continuous control of associated gait devices.

### SUMMARY

The invention is directed to control systems and methods for gait devices. In one aspect of the invention, a method for controlling gait devices includes the steps of measuring kinematic and/or loading states of limb or robotic segments; conditioning the resulting state measurement by any combination or order of integration, differentiation, filtering, and amplification; transforming the conditioned state measurement by coordinate transformation; optionally conditioning the transformed state measurements a second time in a manner similar to the first conditioning step; and using the transformed or conditioned transformed state measurements as independent variables in a predetermined reference function to calculate a desired reference command for any number of actuators.

### BRIEF DESCRIPTION OF THE DRAWINGS

In the drawings, wherein like reference numerals refer to similar components:

FIG. 1 is a schematic representation of a method for controlling gait devices;

FIG. 2 is a perspective view of a gait augmentation robot, showing an example of coordinate systems of kinematic states;

FIG. 3 is a schematic representation of a second method for controlling gait devices;

FIG. 4A is a front view of a representation of an amputee having a prosthesis;

FIG. 4B is a perspective view of the amputee shown in FIG. 4A, showing a coordinate system used for controlling gait devices; and

FIG. 5 is a schematic representation of a control system, implementing a method for controlling gait devices.

### DETAILED DESCRIPTION

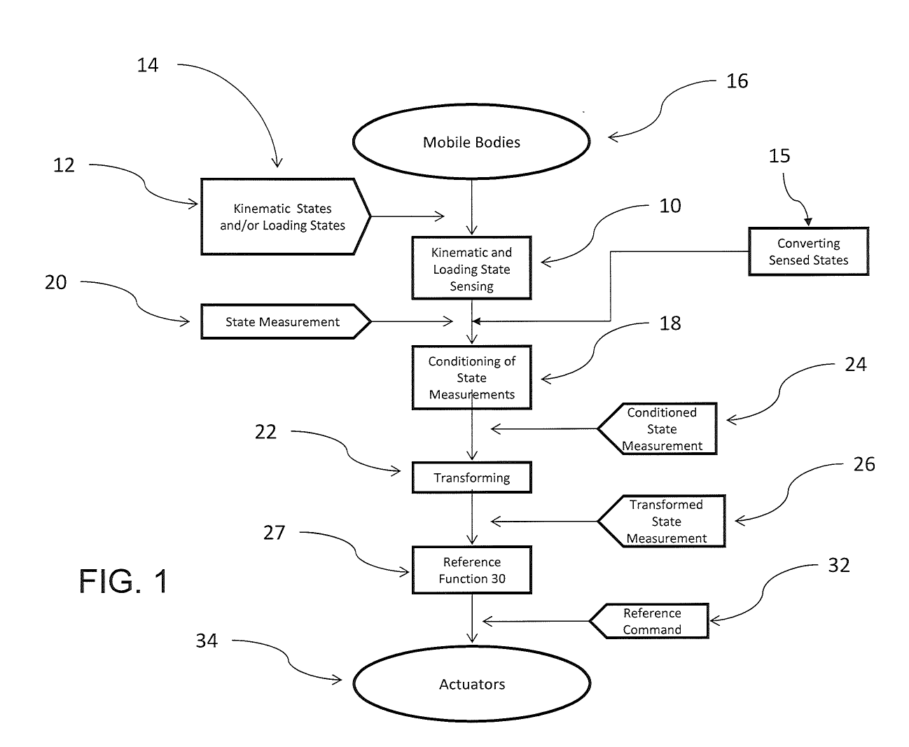

Referring to FIG. 1, a method for controlling gait devices includes: sensing 10 kinematic states 12 and/or loading states 14 generated by mobile bodies 16, converting 15 sensed states into state measurements 20, conditioning 18 state measurements 20 to yield conditioned state measurements 24, transforming 22 the conditioned state measurements 24 into transformed state measurements 26, and inputting 27 the transformed state measurements 26 into a reference function 30 to derive a reference command 32. The reference command 32 is then used by one or more actuators 34 to aid in control of one or more gait devices (not shown).

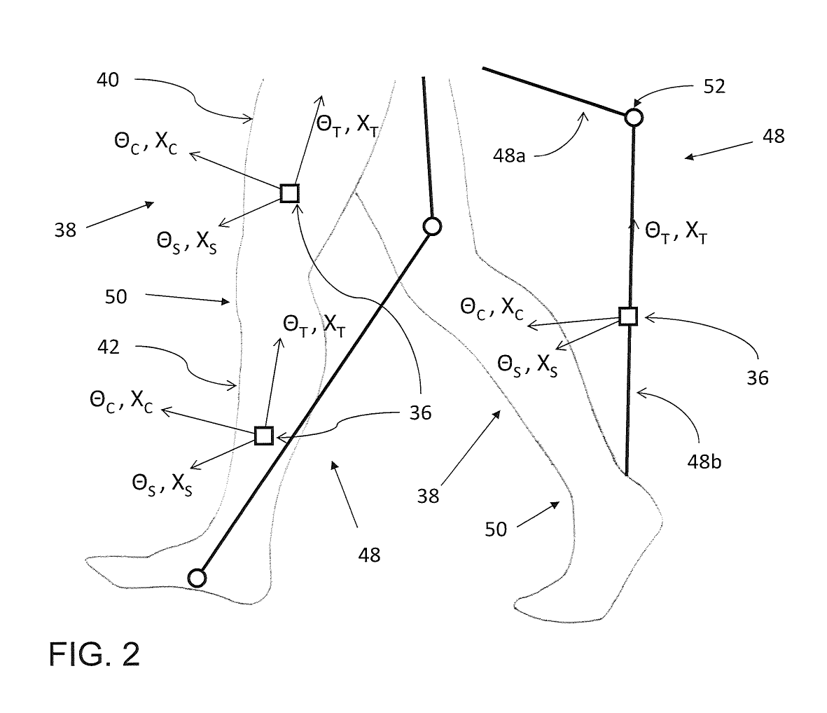

As used herein, the term “mobile body” is defined as a limb segment or robotic segment. As used herein, the term “kinematic state” used in connection with a mobile body, is defined as an angular position, linear position, linear velocity, angular velocity, linear acceleration, or angular acceleration associated with a mobile body with reference to a fixed world frame or a frame fixed to any other mobile body. Referring to FIG. 2, the kinematic state 12 can be measured using any type of sensor(s) 36 or sensor system affixed to limb segments 38, such as thigh segments 40 or shank segments 42 of human legs, for example. Sensors 36 can also be affixed to robotic segments 48, which may include multiple segments. FIG. 2 shows robotic segments 48 having upper segments 48a and lower segments 48b.

The sensors 36 are configured to measure velocities, accelerations, angular positions and/or linear positions in coordinate frames, which are oriented with the limb segment or robotic segment to which they are affixed. These coordinate frames have three orthogonal axes: (1) the sagittal axis (\textcircled{−}_{S}, X_{S}), (2) the coronal axis (\textcircled{−}_{C}, X_{C}), and (3) the transverse axis (\textcircled{−}_{T}, X_{T}). The sagittal axis (\textcircled{−}_{S}, X_{S}) is oriented normal to the sagittal plane of the segment, while the coronal axis (\textcircled{−}_{C}, X_{C}) is oriented normal to the coronal plane of the segment and the transverse axis (\textcircled{−}_{T}, X_{T}) is oriented normal to the transverse plane of the segment. As such, each sensor 36 is oriented so that its axis of measurement is any linear combination of three unit vectors in the direction of the sagittal, coronal, and transverse axes.

As used herein, the term “loading state” used in connection with a mobile body, is defined as a moment or force experienced by a mobile body. Also referring to FIG. 2, a moment or force can be measured, using any type of sensor 36 or sensor system. The sensors 36 can be located on one or more limb segments 38, robotic segments 48, limb joint 50, robotic joint 52, or any type of limb-robot interface.

With regard to the loading state 14, the sensors 36 measure force or moment experienced at the point in the limb or robot in coordinate frames, where the coordinate frames are defined by the sagittal, coronal, and transverse axes. Each sensor 36 is also oriented so that its axis of measurement is any linear combination of the three unit vectors in the direction of the sagittal, coronal, and transverse axes.

Referring back to FIG. 1, after sensing of the kinematic states 12 and/or loading states 14 by sensors 36 or sensor system, converting 15 of the sensed states occurs. In this step, the sensed states are converted from an output of the sensor 36 to a desired unit of measurement that yields a state measurement 20.

State measurements 20 are then conditioned to yield conditioned state measurements 24. Conditioning 18 is realized by any filtering method, including, but not limited to Kalman filtering, transfer function use, integration, differentiation, and amplification. These filtering methods may be performed as many times as desired.

Amplification may result from a gain of any nonzero number, including by a unity gain. In addition, conditioning may also be realized by any combination and order of filtering, integration, differentiation, and/or amplification. Filtering can be employed for multiple uses, including but not limited to: reduction of noise in state measurements, reduction of inaccuracies in state measurements, or alteration of state measurements. For example, alteration of state measurements may be performed in a manner similar to integration or differentiation such that drift in numerical integration or noise in numerical differentiation is eliminated.

Transforming 22 of conditioned state measurements 24 is generally described as changing coordinate systems to yield transformed state measurements 26, which are realized by isometric or non-isometric transformations. These types of transformations include rotations and dilations. Other types of transformations, however, include identity transformations, projections, changes to other coordinate systems, and changes of scale. The projections may either be orthogonal or oblique. In addition, other coordinate systems may include polar coordinate systems, barycentric coordinate systems, and other similar types of coordinate systems. Changes of scale may be log scale or any other function of scale. Moreover, these transformations may include any transformation where the transformed state measurements are any mathematical function of the conditioned state measurements; or any combination in any order of transformations, projections, changes of coordinate system, changes of scale, mathematical functions, etc.

A transformed state measurement coordinate system is not restricted to have the same number of dimensions as the conditioned state measurement coordinate system. In fact, there may be more or less transformed state measurements than conditioned state measurements. Transformation is generally employed so that a robust relationship between the conditioned state measurements and the desired output reference command can be found. However, transformation is not limited to this use.

Transformed state measurements 26 are used as arguments to one or more reference functions 30. The transformed state measurements 26 are therefore used to calculate reference commands 32, using the reference functions 30. Each reference function 30 is a function that relates the transformed state measurements 26 as independent variables to the reference command as a dependent variable. The reference function 30 can be represented in any way that accepts inputs and that outputs a unique value for each combination of inputs. As such, the reference function may be represented using any suitable method. Suitable methods of representation include look up tables, mathematical functions, or combinations of tables and mathematical functions.

The reference function 30 is determined by recording data from sensors 36 and then by using the aforementioned method(s) to obtain the transformed state measurements 26 combined with either a recording or calculation of a desired reference command. The reference function 30 is also made to match data from one or more gait activities. Such activities include as walking, running, traversing slopes or stairs, avoiding obstacles, and other similar activities.

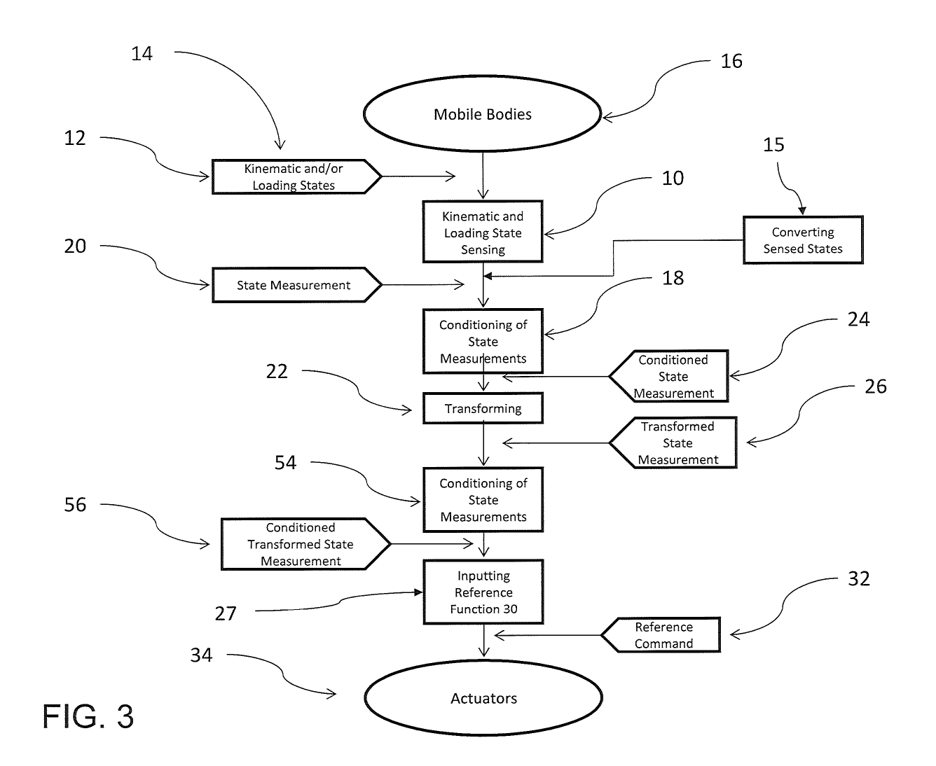

As shown schematically in FIG. 3, after transforming 22 of the conditioned state measurements 24, one or more of the transformed state measurements 26 may be conditioned in an additional conditioning step 54. This step occurs before the transformed state measurements are used as arguments for the reference function 30. In this conditioning step 54 conditioned transformed state measurements 56 result. Here, conditioning may also be realized by any filtering method. Such filtering methods include, but are not limited to Kalman filtering, transfer function use, integration, differentiation, and amplification. Integration and differentiation may be performed as many times as is desired; while amplification may result from a gain of any nonzero number, not including a unity gain.

Filtering method(s) may include filtering, integration, differentiation, and/or amplification performed in any combination and in any order. Any transformed state measurements and conditioned transformed state measurements are used as arguments to one or more previously determined reference command functions. These measurements are then used to calculate the desired reference commands. Each reference command function is a function that relates the transformed state measurements and the conditioned transformed state measurements as independent variables to the desired reference command as a dependent variable. The reference command function is made to match data from any combination of two or more gait activities such as walking, running, traversing slopes or stairs, obstacle avoidance, or similar activities.

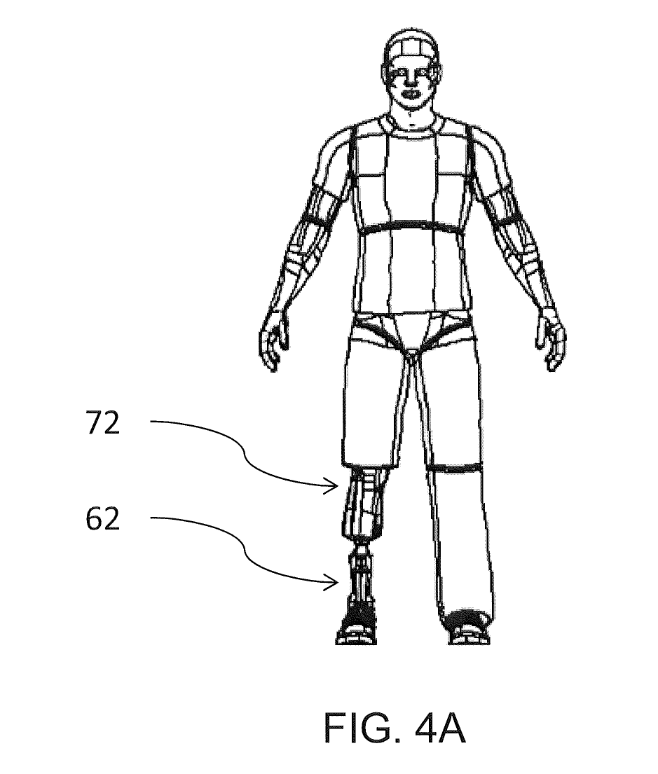

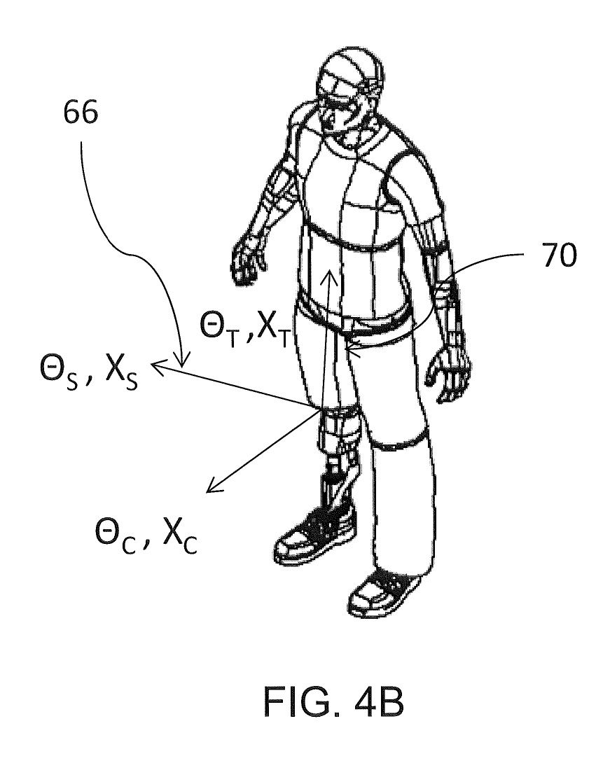

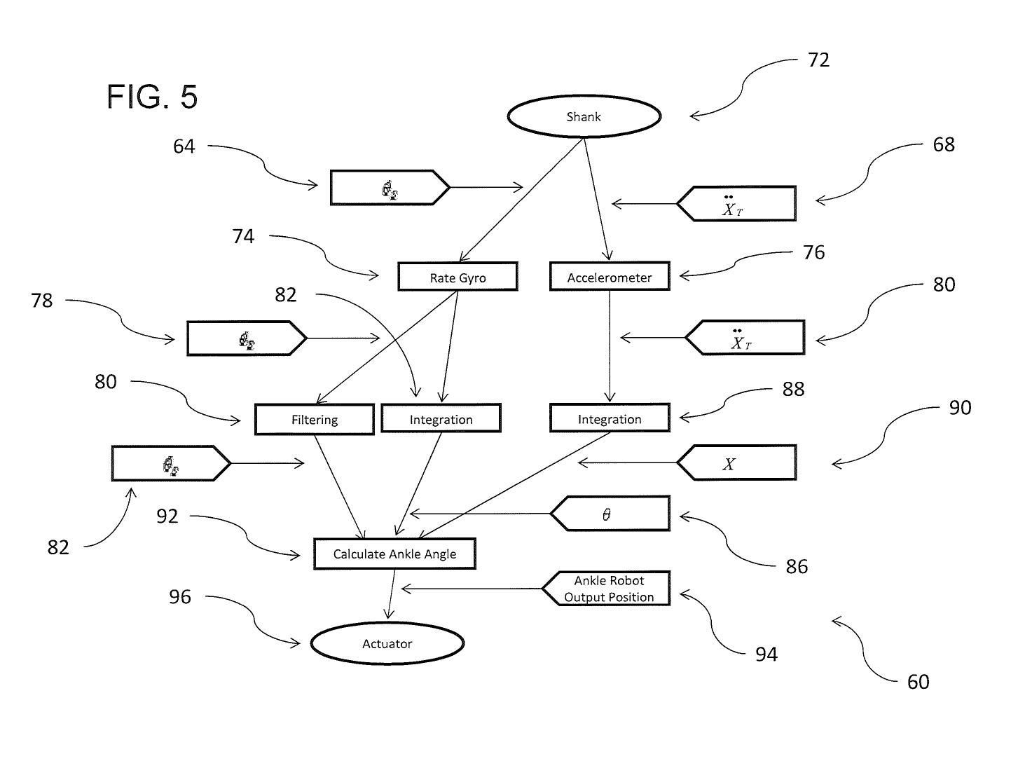

Referring to FIGS. 4A, 4B, and 5, an implementation of the method is shown as a control system 60 for an ankle prosthesis 62. In the control system 60, an angular velocity kinematic state 64 in the sagittal direction 66 and an acceleration kinematic state 68 in the transverse direction 70 of a shank 72 are measured. In this implementation, measurements are taken using a rate gyro 74 and an accelerometer 76, respectively, to yield an angular velocity state measurement 78 and an acceleration state measurement 79.

The angular velocity state measurement 78 is conditioned by filtering 80 to yield an angular velocity conditioned state measurement 82, while the angular velocity state measurement 78 is conditioned by integration 84 to get an angle conditioned state measurement 86, and the acceleration state measurement 79 is conditioned by double integration 88 to yield a position conditioned state 90. The angular velocity conditioned state measurement 82, angle conditioned state measurement 86, and position conditioned state measurement 90 are each transformed by identity transformation (not shown) resulting in no change to the conditioned state measurements 82, 86, 90. The conditioned state measurements 82, 86, 90 are then used as arguments in the ankle angle reference command function 92 which yields an ankle robot output position reference command 94. The command function 94 is then used by the actuator 96 of the ankle prosthesis 62.

The control systems and methods for gait devices described herein have several benefits. For example, the continuous nature of the reference command calculation is beneficial because the method continuously measures a limb or robot segment directly and computes a reference command from a continuous differentiable function. As a result, the reference command is less likely to make sudden jumps or undesirable oscillations. Moreover, because the reference command is a function of measured quantities, generally there is no decision making and no state machine switching of states. Dealing with decision making and state transitions is known to be error prone, often resulting in undesirable operation when a state is chosen incorrectly.

The aforementioned control systems and methods may be employed in a wide field of applications. Some examples, which are in no way exhaustive, include controlling lower limb prostheses and orthotic devices and assisting in the operation of exoskeleton devices. Also, the method may be employed in computer animation, gaming, and other fields where the control of robotic and bionic machines benefit from characterization of cyclic patterns.

Thus, control systems and methods for controlling gait devices are disclosed. While embodiments of this invention have been shown and described, it will be apparent to those skilled in the art that many more modifications are possible without departing from the inventive concepts herein. The invention, therefore, is not to be restricted except in the spirit of the following claims.

What is claimed is:

1. A method for controlling gait devices, wherein gait devices are robotic devices worn by a user to replace limbs or assist movement, the method comprising:
Measuring, by use of one or more sensors, one or more physical states of one or more mobile bodies, wherein each mobile body comprises a limb segment or robot segment;
conditioning the measured physical state(s);
transforming the conditioned physical state(s); and
generating a reference command for control of one or more actuators using commands derived from the input of the transformed physical state(s) into a reference function, wherein the reference function is based on at least one gait activity.

2. The method of claim 1, wherein at least one of the physical states is a kinematic state, wherein a kinematic state is defined as angular position, linear position, linear velocity, angular velocity, linear acceleration, or angular acceleration.

3. The method of claim 1, wherein at least one of the physical states is a loading state, wherein a loading state is defined as a moment or force applied to or internal to a limb segment or robot segment.

4. The method of claim 1, wherein the physical states are made up of any combination of kinematic or loading states.

5. The method of claim 1, wherein the sensors are coupled to limb segments.

6. The method of claim 1, wherein the sensors are coupled to robotic segments.

7. The method of claim 1, wherein conditioning is realized by one or more conditioning method selected from the group consisting of Kalman filtering, use of a transfer function, integration, differentiation, amplification by a non-zero gain, and addition of a constant offset.

8. The method of claim 1, wherein transformation is realized by one or more transformation method selected from the group consisting of rotations, dilations, orthogonal or oblique projections, the identity transformation, changes of coordinate systems, changes of scale, and mathematical functions.

9. The method of claim 1, wherein the steps consisting of conditioning and transformation are reversed.

10. A method for controlling gait devices, wherein gait devices are robotic devices worn by a user to replace limbs or assist movement, the method comprising:
Measuring by use of one or more sensors one or more physical states of one or more mobile bodies, wherein each mobile body comprises a limb segment or a robot segment;
conditioning the measured physical state(s);
transforming the conditioned physical state(s);
conditioning the transformed physical state(s); and
generating a reference command for control of one or more actuators using commands derived from the input of the conditioned transformed physical state(s) into a reference function, wherein the reference function is based on at least two gait activities.

11. The method of claim 10, wherein at least one of the physical states is a kinematic state, wherein a kinematic state is defined as angular position, linear position, linear velocity, angular velocity, linear acceleration, or angular acceleration.

12. The method of claim 10, wherein at least one of the physical states is a loading state, wherein a loading state is defined as a moment or force applied to or internal to a limb segment or robot segment.

13. The method of claim 10, wherein the physical states are made up of any combination of kinematic or loading states.

14. The method of claim 10, wherein the sensors are coupled to limb segments.

15. The method of claim 10, wherein the sensors are coupled to robotic segments.

16. The method of claim 10, wherein conditioning is realized by one or more conditioning method selected from the group consisting of Kalman filtering, use of a transfer function, integration, differentiation, amplification by a non-zero gain, and addition of a constant offset.

17. The method of claim 10, wherein transformation is realized by one or more transformation method selected from the group consisting of rotations, dilations, orthogonal or oblique projections, the identity transformation, changes of coordinate systems, changes of scale, and mathematical functions.
